(19) United States
(12) Patent Application Publication
(10) Pub. No.: US 2006/0240058 A1
(43) Pub. Date: Oct. 26, 2006

(54) Combinatorial surface chip compositions for selection, differentiation and propagation of cells

(76) Inventors: Zorina S. Galis, Indianapolis, IN (US); Carson J. Meredith, Marietta, GA (US)

Correspondence Address: E.J. ASBURY III, LLC, 3330 CUMBERLAND BOULEVARD, SUITE 500, ATLANTA, GA, 30339-5985, US

(21) Appl. No.: 10/553,642

(22) PCT Filed: Apr. 16, 2004
(86) PCT No.: PCT/US04/11970

§ 371 Date: Oct. 14, 2005

Related U.S. Application Data
(60) Provisional application No. 60/463,752, filed on Apr. 16, 2003.

(51) Int. Cl. C40B 30/06; A61F 13/00

(52) U.S. Cl. 424/422; 435/7.1

(57) ABSTRACT

A combinatorial library high-throughput technique for varying polymer surface structures has been developed that allows rapid, efficient, and accurate investigation of the effects of microstructure, roughness, and surface chemistry on stem cell adhesion, proliferation and differentiation. Bloactive molecules, such as cytokines, growth factors, and extracellular matrix components can be attached to the surfaces to bind to and/or modify the cell characteristics.

### CROSS-REFERENCE TO RELATED APPLICATIONS

This application claims the benefit of U.S. Provisional Application No. 60/463,752, filed Apr. 16, 2003.

### BACKGROUND OF THE INVENTION

The present invention generally relates to substrates having different properties and/or molecules immobilized thereon, which are useful for selection, differentiation, and propagation of cells, especially stem cells.

### Current Methodologies for Stem Cell Culture and Differentiation

The current procedures employing the therapeutic potential of stem cells continue to remain more an “art”, which pose great problems with reproducibility and do not allow the reliability necessary for clinical applications. Thus, the precise design and standardization of conditions used for isolation, expansion, and differentiation of stem cells is desired. The term “stem cells” as used herein, refers to both committed and uncommitted stem cells.

Currently available technologies for in vitro expansion of hematopoietic stem cells (HSC) can be divided into stroma-based and stroma-free methodologies. Stroma-based protocols involve culturing HSC on “feeder layers” derived from bone marrow stromal cells. From a clinical standpoint, this approach may be unsuitable for expansion of peripheral blood stem cells (PBSC) for autologous transplantation, due to issues of cross-contamination and immune activation when PBSC from one donor are cultured on stromal cells derived from an unrelated human source.

A growing literature indicates that the adhesion, migration, proliferation, and differentiation of numerous cell types can be modulated by the underlying substrate properties, including surface chemistry, microstructure, and roughness. These differences in cell function are mediated by alterations in adhesive interactions primarily involving integrin receptors. Previous analyses have demonstrated that adhesive interactions play important roles in bone marrow-derived stem cell proliferation and differentiation. The bone marrow presents a complex microenvironment in which the growth, differentiation, and self-propagation of hematopoietic stem cells are regulated by soluble and matrix-associated cytokines and by adhesive interactions with accessory stromal cells and the underlying extracellular matrix (Arroyo, A. G. et al. Immunity 11(5): 555-566 (1999); Kollet, O. et al. Blood 97(10): 3283-3291 (2001)). Structural and functional compartmentalization of this microenvironment accounts for the amazing capacity of the bone marrow to generate a large diversity of mature cell types in response to the body's needs. Recent research indicates that interactions between hematopoietic progenitor cells and the supporting stroma are mediated by several families of adhesion molecules, including integrins, selecting, sialomucins, and members of the IgG superfamily (Ogawa, M. et al. Blood 93(4): 1168-1177 (1999); Peled, A. et al. Blood 95(11): 3289-3296 (2000)). In particular, integrin-mediated adhesive interactions play important regulatory roles in hematopoiesis. For human hematopoietic progenitors, integrins α4β1 (VLA-4), α5β1 (VLA-5), and αLβ2 (LFA-1) are involved in homing of HSC to the bone marrow and adhesion to the stroma (Robledo, M. M. et al. J. Biol. Chem. 273(20): 12056-12060 (1998); Voermans, C. et al. Stem Cells 18(6): 435-443 (2000); Williams, D. A. et al. Nature 352: 438-441 (1991); Levesque, J. P. et al. J. Exp. Med. 181(5): 1805-1815 (1995); Zweegman, S. et al. Exp. Hematol. 28(4): 401-410 (1995); Xie, B. et al. J. Biol. Chem. 273(19): 11576-11582 (1998); Yokota, T. et al. Blood 91(9): 3263-3272 (1998); Schofield, K. P. et al. Blood 91(9): 3230-3238 (1998)). Moreover, integrin binding, in concert with hematopoietic growth factors, activates intracellular signaling pathways regulating hematopoietic progenitor cell survival, proliferation, and differentiation (Robledo, M. M. et al. J. Biol. Chem. 273(20): 12056-12060 (1998); Wang, M. W. et al. Cell Growth Differ. 9(2): 3230-3238 (1998); Strobel, E. S. et al. Blood 90(9): 3524-3532 (1997); Levesque, J. P. et al. Blood 88(4): 1168-1176 (1996); Hurley, R. W. et al. J. Clin. Invest. 96(1): 511-519 (1995); Hurley, R. W. et al. Exp. Hematol. 25(4): 321-328 (1997); Herrera, R. et al. Exp. Cell Res. 238(2): 407-414 (1998)). In CD34⁺ cells, for example, binding of integrins α4β1 and α5β1 to fibronectin in the presence of pharmacological concentrations of several cytokines promotes cell proliferation, while integrin engagement under low cytokine concentrations blocks cell cycle progression (Jiang, Y. et al. Blood 95(3): 846-854 (2000); Jiang, F. et al. Blood 99(10): 3579-3584 (2002); Gotoh, A. et al. Ann. Hematol. 75(5-6): 207-213 (1997)). Although several integrin/growth factor-regulated signaling molecules, such as PI-3 kinase, Src, and Ras/ERK members, appear to play key roles in integrin activation/adhesion and subsequent proliferative and differentiation events (Becker, P. S. et al. Exp. Hematol. 27(3): 533-541 (1999); Liang, X. J. Biol. Chem. 277(16): 13732-13738 (2002); Klingmuller, U. et al. Proc. Natl. Acad. Sci. USA 94(7): 3016-3021 (1997); Kapur, R. et al. Blood 97(7): 1975-1981 (2001); Wang, J. F. et al. Blood 95(8): 2505-2513 (2000); Aota, S. et al. J. Biol. Chem. 269(40): 24756-24761 (1994); Garcia, A. J. et al. Biochem. 41: 9063-9069 (2002): Moursi, A. M. et al. J. Cell Sci. 110(Pt18): 2187-2196 (1997)), the molecular mechanisms governing these responses remain poorly understood.

Given the complex interactions between substrate physical and chemical properties, integrins and other adhesion molecules, and signaling events, there is a distinct need for a novel “screening” technology for developing optimal substrate surfaces for stem cell expansion, survival, and differentiation.

In addition, there is a need for a method to direct the differentiation of stem cells into specific lineages, such as endothelial, neuronal or hematopoietic lineages, which would open the possibility of stem cell-based engineering strategies to treat, repair, or replace tissues and organs damaged by disease or chemotherapy. The major practical limitations in using stem cells in therapeutics have been the lack of robust approaches to (1) identify, select, and enrich stem cells from complex mixtures of cells; (2) expand rare pluripotent stem cells under conditions that retain self-renewal capacity; and (3) selectively direct differentiation of pluripotent stem cells toward specific tissue lineages.

### Vascular Regeneration

One possible application of a stem cell-based engineering strategy is in the repair or regeneration of the vasculature. Blood vessels play an essential role in the survival and function of all tissues in the body. Endothelial cells (EC), which line the inside of blood vessels, play a key role in the vasculature since they maintain the non-thrombogeneic interface with blood, regulate the selective transfer of molecules and cells between blood and tissues, and control the vasoactive response of blood vessels to changes in the hemodynamic environment. In the embryo, EC arise from precursors called angioblasts or haemangioblasts, while in the adult, they are differentiated from circulating endothelial progenitor cells (EPC). The endothelial cells by themselves are enough to form the simplest type of blood vessels, known as capillaries. Larger diameter blood vessels, such as arterioles, contain additional cell types, including smooth muscle cells and pericytes.

Any defect in the function of blood vessels, such as may occur by wounding or following a vascular clinical event, such as heart attack or stroke, will render the supplied tissues non-functional. Depending on the location and size of the defects, blood vessel failure is often life-threatening. Recent studies have demonstrated that environmental cues lead to the recruitment of circulating endothelial progenitor cells to tissues that have become ischemic (those that lost their natural blood supply) due to injury. These cues also appear to initiate the differentiation of the EPCs. Although the exact identity of the cues remains unknown, factors shown to be essential in the differentiation of progenitors into vascular cells and the creation of the identity of arteries, capillaries, and veins include cell interactions with the matrix scaffold, presence of growth factors, and hemodynamics.

Thus, a very desirable therapeutic strategy would involve directing the differentiation of hematopoietic stem cells into endothelial progenitor cells followed by therapeutic administration of the EPCs to restore function to ischemic tissues.

It is therefore an object of the invention to direct stem cell differentiation to provide stem cell-based engineering strategies to treat, repair, or replace tissues and organs damaged by disease or chemotherapy.

It is another object of the invention to provide approaches for expanding stem cells and endothelial progenitor cells (EPC) in vitro.

It is still another object of the invention to provide methods for directing stem cell and EPC function by controlling adhesive interactions through the underlying substrate chemistry, microstructure, and roughness.

Another object of the present invention is to provide a rational approach to engineer and discover surfaces that direct stem cell and EPC survival, expansion, and differentiation.

### SUMMARY OF THE INVENTION

Multi-component combinatorial surfaces of varying physicochemistry and microstructure have been developed which are useful for manipulation of cells attached to the surfaces. For example, thermally-controlled phase separation of biocompatible polymer blends, such as poly(D,L-lactide), PDLA, and poly(ε-caprolactone) (PCL) can be used to generate surfaces with chemically distinct, heterogeneous microdomains enriched in one or more properties or molecules. Various substrate compositions can be used to modulate cell adhesion, proliferation, and differentiation. Examples of bioactive molecules that can be attached to the substrate to bind to and/or modify the cell characteristics include growth factors, cytokines and extracellular matrix molecules.

The combinatorial surfaces are useful for screening for, or selection of, cells which bind to the different microdomains and/or bioactive molecules. While any type of cell may be used with the surfaces, preferred cell types to be modified include stem cells and endothelial progenitor cells.

Stem cell or undifferentiated cell survival and self-renewal can be modulated by controlling adhesive interactions through the underlying substrate. Multi-component combinatorial surfaces (Combi-chips) of varying physicochemistry and microstructure can be used to analyze the effects of a wide range of surface properties on EPC adhesion, survival, and proliferation, which can then be selected based on outcome. Progenitor cells are cultured on engineered substrates and adhesion, survival and proliferation can be analyzed by measuring anti-apoptotic signals and biological markers of growth, proliferation, and differentiation. Stem-cell-preserving substrates will induce anti-apoptotic signals such as activation of focal adhesion kinase (FAK), bcl-2 family members and PI3-kinase, while stem cell proliferation will be associated with up-regulation of growth signals such as Myc and the Ras/ERK pathway. Specific surface chemistries and formulations (composition/microstructure) that support enhanced stem cell adhesion, survival, proliferation, and preliminary EPC differentiation may be identified. The small subset (4-6 surfaces) of candidate substrates can then be analyzed separately for differentiation and in vivo functional outcomes. In parallel, evaluations of these candidate substrates can be used to engineer second-generation surfaces for enhanced control of stem cell adhesion and function.

The substrates are useful in the identification of surfaces, structures, compositions, and/or ligands thereon, to control the propagation, growth and differentiation of cells cultured thereon. One advantage of the combinatorial method is that it can be used to identify surface properties that specifically reduce or eliminate the need to add biological molecules to culture systems. This is very attractive due to the current regulatory issues with biological materials, specifically human-derived products.

The substrates can also be used to differentiate a population of cells, especially stem cells, where the different microdomains and/or bioactive molecules are sued to induce differentiation and/or propagation of the cells in one or more different ways. In one embodiment, the surfaces are used to direct the differentiation of stem cells into specific lineages, such as endothelial, neuronal or hematopoietic lineages, and used to treat, repair, or replace tissues and organs damaged by disease or chemotherapy. In a preferred embodiment, stem cells are directed to differentiate into endothelial progenitor cells (EPC), due to their potential to promote the formation of vascular structures that can rescue and render functional an ischemic organ, graft, or tissue engineered construct.

### BRIEF DESCRIPTION OF THE DRAWINGS

FIGS. 1A, 1B and 1C are schematics of the continuous composition gradient deposition process, showing the gradient column (FIG. 1A), deposit of a stripe (FIG. 1B), and spreading of the film (FIG. 1C).

FIGS. 2A, 2B and 2C are microstructural and roughness characterizations of a PDLA/PCL library: FIG. 2A is a three-dimensional graph of diameter of the PCL-rich regions (microns) versus temperature (° C.) versus φ_{PCL} (mass fraction); FIG. 2B is a three-dimensional graph of roughness (nm) versus temperature (° C.) versus φ_{PCL} (mass fraction); and FIG. 2C is a three-dimensional graph of surface fraction PCL versus temperature (° C.) versus φ_{PCL} (mass fraction).

FIG. 3 is a three-dimensional graph of quantitative cell density analysis of mouse bone marrow cells cultured on a combinatorial chip, plotting annealing temperature (° C.) versus PCL (mass fraction) versus average cell density.

### DETAILED DESCRIPTION OF THE INVENTION

Phase-separation can be used to prepare porous three-dimensional scaffolds for tissue engineering or drug delivery. The combinatorial approach has been extended to investigate adhesion, proliferation, and differentiation of biological cells as a function of surface-patterned microstructures. Phase-separated libraries of biodegradable polymers can be cultured with cells, exposing the cells to a wide variety of surface features in a single experiment. These combinatorial “chips” (also referred to as “combi-chips”) contain thousands of surface features of varying chemistry, microstructure, and topography.

In a preferred embodiment, stem cell adhesion, expansion, and commitment to differentiated phenotypes can be regulated through the chemistry and physical properties of the underlying substrate. The term “stem cells” as used herein, refers to both committed and uncommitted stem cells. For example, uncommitted stem cells may be driven to differentiate based upon the chemical and physical properties of the above-described underlying substrate. Such differentiation may result in the production of committed stem cells.

Synthetic and hybrid substrates may be engineered to control progenitor cell adhesion, maintenance, self-renewal, and differentiation. Well-defined substrates that control stem cell expansion and differentiation can lead to robust stem cell-based strategies for the treatment of diseased tissues and organs. The frequency and function of these progenitor cell populations from the bone marrow and the circulating pool can be compared to establish potential differences in activities. Enhancement of EPC numbers and functionality using ex vivo expansion and/or in vivo administration of colony stimulating factors can result in improvement of vascularization and restoration of function in ischemic tissues.

I. Combinatorial Surfaces (Combi-Chips)

Gradient combinatorial polymer surface libraries can contain property gradients that cover thousands of compositions, annealing temperatures, and surface structures. Bioactive molecules, such as growth factors, cytokines and extracellular matrix molecules, can also be attached to the surface to bind to and/or modify cell characteristics.

A. Preparation of the Combinatorial Surfaces

Libraries of phase-separated polymers with systematic variations in composition (φ) can be prepared using a solvent casting procedure that results in a controllable gradient in blend composition. Two computer-controlled pumps inject polymer solution A into a vial initially containing polymer solution B, while withdrawing the mixture into a pump to create a composition gradient. Polymer solutions A and B may comprise any of the polymers described below. This gradient is then painted as a thin stripe and spread onto a microscope slide. Flow remains in the laminar regime to prevent turbulent mixing, and FTIR characterization confirms the presence of linear, controllable composition gradients. The resulting linear φ-gradient sample is annealed over an orthogonal temperature gradient using a custom heating stage to create T, φ-gradient libraries. The combi-chips are characterized in terms of phase behavior, microstructure, surface roughness, and chemistry using optical, AFM, and FTIR microscopes fitted with automated sample stages.

Cells can then be cultured on the surface of pre-annealed T, φ libraries which have been quenched to room temperature and sterilized in 70% ethanol. Each library allows cells to be exposed to approximately 1000 distinct chemistry, microstructure, and roughness combinations in a single experiment. The libraries can be analyzed in terms of cell adhesion, viability, and proliferation using histochemical methods known in the art as a function of surface chemistry, microstructure, and roughness. These relationships allow for efficient selection of the most relevant T, φ conditions for detailed characterization of materials, e.g. those with the strongest positive or negative effects on cell function.

In addition to using phase separation phenomena as a mechanism for generating patterned surfaces, other techniques can be used as well. These include using chemical reactions to alter structure and surface chemistry. For example, multicomponent copolymers (block, alternating, and random) can be synthesized in situ on the libraries (Sormana, J. L. and Meredith, J. C. Macromolecules, 37, 2984 (2004)) to create segregated chemical patterns from nanometers to micrometers in size. Inorganic and organic particulate fillers, crosslinkers, and bioactive molecules can also be blended into the library during deposition. In addition, surface modification via adsorption, ion exchange, or chemical reactions can be utilized to attach bioactive molecules (integrin ligands for example) to one other surface phases.

B. Polymers Used in the Combinatorial Surfaces

Polymeric materials that may be used in the preparation of the combi-chips include synthetic polymers or polymer blends as well as purified biological polymers or polymer blends. Appropriate synthetic polymers include without limitation polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, ethylene vinyl acetates, polysulfones, nitrocelluloses and similar copolymers.

These synthetic polymeric materials can be woven or knitted into a mesh to form a matrix or substrate. Alternatively, the synthetic polymer materials can be molded or cast into appropriate forms.

In one embodiment, the synthetic polymers are nondegradable, for example segmented poly(urethane ureas).

In another embodiment, the polymer compositions comprise blends of biodegradable materials, for example poly(D,L-lactide), poly(L-lactide), poly(D-lactide-co-glycolide), or poly(caprolactone).

Suitable polymers are commercially available. Natural or modified polymers can also be used. Suitable biological polymers include, without limitation, collagen, elastin, silk, keratin, gelatin, polyamino acids, cat gut sutures, polysaccharides (e.g., cellulose and starch) and copolymers thereof.

Recombinant DNA technology can be used to engineer virtually any polypeptide sequence and then amplify and express the protein in either bacterial or mammalian cells.

Polymers can be appropriately formed into a substrate by techniques such as weaving, knitting, casting, molding, extrusion, cellular alignment and magnetic alignment. For a description of magnetic alignments see, for example, R. T. Tranquillo et al., Biomaterials 17:349-357 (1996).

C. Definition of Microdomains

Each distinct chemistry, microstructure, and roughness combination defines a microdomain of the combi-chip. The number of microdomains, or discrete regions, is determined by the technique used to manufacture the chip, and the dimensions of the chip.

A microdomain can be characterized as any structure or surface region with size from 1 nm to 100 μm that exhibits a difference in at least one of the following properties from its immediate surroundings: roughness, bulk chemistry, surface chemistry, crystallinity, phase (liquid or solid), lateral dimensions, and shape.

A microdomain may include structures or specific regions on the surface with lateral dimensions from several nanometers up to hundreds of micrometers. The lateral structures may be a separate phase from the surrounding matrix, induced by liquid-liquid demixing, by block-copolymer segregation, or by liquid-solid phase separation, as in crystallization and in soluble fillers. The microdomains may exhibit differences in roughness. Roughness values (root-mean-square) of microdomains will typically fall in the range of 0.1 nm to 50 μm. The microdomains also may exhibit differences in chemistry and shape from the surrounding environment.

D. Manufacture of Microdomains

Any naturally-occurring or externally-induced phenomenon that creates or alters lateral dimensions, chemistry, roughness, patterning, crystallinity, or shape of structures on a size-scale from 1 nm to 100 mm can be, in principal, utilized to manufacture microdomains. For example, lower critical solution temperature (LCST) and upper critical solution temperature (UCST) liquid-liquid phase separation are common methods. The primary surface differences between regions within and outside the LCST are the microstructure (lateral distribution of chemically distinct domains) and surface roughness. Phase separation also induces changes in the roughness of the surface, which are attributed to surface tension differences between chemically distinct domains and between crystalline and amorphous polymer forms.

Other methods for microdomain manufacture include the following, either alone or in concert with others: microlithography, micro contact printing, nanolithography, electron beam lithography, inorganic and organic fillers, crystallization, and physical molding.

II. Bioactive Molecules to be Bound to Substrate with Microdomains

Bioactive molecules, as used herein, refer to molecules that bind to cell surface receptors and regulate the growth, replication or differentiation of target cells or tissue. Preferred molecules are growth factors, cytokines and extracellular matrix molecules. Examples of growth factors include epidermal growth factor (EGF), platelet-derived growth factor (PDGF), transforming growth factors (TGFα, TGFβ), hepatocyte growth factor, heparin binding factor, insulin-like growth factor I or II, fibroblast growth factor, erythropoietin, nerve growth factor, bone morphogenic proteins, muscle morphogenic proteins, and other factors known to those of skill in the art. Additional growth factors are described in “Peptide Growth Factors and Their Receptors I” M. B. Sporn and A. B. Roberts, eds. (Springer-Verlag, New York, 1990), for example.

Growth factors can be isolated from tissue using methods known to those of skill in the art. For example, growth factors can be isolated from tissue, produced by recombinant means in bacteria, yeast or mammalian cells. In addition, many growth factors are also available commercially from vendors, such as Sigma Chemical Co. of St. Louis, Mo., Collaborative Research, Genzyme, Boehringer, R&D Systems, and GIBCO, in both natural and recombinant forms.

Examples of cytokines include the interleukins and granulocyte-monocyte-colony stimulating factor (GM-CSF). These are also described in the literature and are commercially available.

Examples of extracellular matrix molecules include fibronectin, laminin, collagens, and proteoglycans. Other extracellular matrix molecules, are described in Kleinman et al. (1987) or are known to those skilled in the art.

Collagen IV-coated surfaces promote differentiation of mouse embryonic stem cells to the endothelial lineage more effectively than do other surfaces including gelatin, fibronectin, and collagen I (Bussolino, F. et al. J. Clin. Invest. 87: 986-996 (1991)). Granulocyte-monocyte colony stimulating factor (GM-CSF) stimulates hematopoietic progenitor cells (Soldi, R. et al. Blood 89: 863-872 (1997), myeloid lineage cells (Aglietta, M. et al. J. Clin. Invest. 83(2): 551-557 (1989), and stromal cells (Bussolino, F. et al. J. Biol. Chem. 264(31): 18284-18287 (1989). In addition, GM-CSF augments EPC mobilization (Takeshita, S. et al. Circulation 90: 11228-11234 (1994); Seiler, C. et al. Circulation 104(17): 2012-2017 (2001)). Human endothelial cells have high-affinity receptors for GM-CSF, which are similar in number and affinity to those on myelo-monocytic cells. GM-CSF exerts a potent stimulatory effect on EPC mobilization, resulting in enhanced neovascularization of severely ischemic tissues as well as de novo vascularization of previously avascular sites.

GM-CSF appears to mobilize significantly greater numbers of the primitive CD34+/CD38−/HLA-DR+ subset of CD34+ cells when compared to G-CSF (Anderlini, P. et al. Bone Marrow Transplantation 21(Suppl 3): S35-39 (1998)). The concurrent administration of GM-CSF and G-CSF is associated with as good a yield of CD34+ cells as G-CSF alone, but with a greater yield of primitive CD34+ subsets. It is expected that these primitive populations are rich in progenitor cells.

III. Cells to be Screened or Cultured

Cells to be screened or cultured using the combinatorial substrates can be any type of cell that will respond to physical and chemical surface features, including most epithelial and endothelial cell types, for example, parenchymal cells such as hepatocytes, pancreatic islet cells, fibroblasts, chondrocytes, osteoblasts, exocrine cells, cells of intestinal origin, bile duct cells, parathyroid cells, thyroid cells, cells of the adrenal-hypothalamic-pituitary axis, heart muscle cells, kidney epithelial cells, kidney tubular cells, kidney basement membrane cells, nerve cells, blood vessel cells, cells forming bone and cartilage, and smooth and skeletal muscle. Cells can be obtained from established cell lines or separated from isolated tissue. The cells used can also be recombinant. Methods for gene transfer are well known to those skilled in the art.

However, while the combinatorial surfaces are generally applicable to any type of cell, preferred cells to be used are stem cells, undifferentiated cells, progenitor cells, and endothelial progenitor cells (EPC).

a. Stem Cells

Both embryonic and adult stem cells can proliferate without differentiating for a long period (a characteristic referred to as long-term self-renewal), and they can give rise to mature cell types that have characteristic shapes and specialized functions. Adult stem cells are rare. Often they are difficult to identify and their origins are not known. Current methods for characterizing adult stem cells are dependent on determining cell surface markers and observations about their differentiation patterns in test tubes and culture dishes. To date, published scientific literature indicates that adult stem cells have been derived from brain, bone marrow, peripheral blood, dental pulp, spinal cord, blood vessels, skeletal muscle, epithelia of the skin and digestive system, cornea, retina, liver, and pancreas. Thus, adult stem cells have been found in tissues that develop from all three embryonic germ layers.

Hematopoietic stem cells from bone marrow are the most studied and are used for clinical applications in restoring various blood and immune components to the bone marrow via transplantation. Murine hematopoietic stem cells can be purified using a combination of cell surface markers such as the stem cell antigen, Sca-1, the receptor tyrosine kinase c-Kit and low or negative levels of lineage markers (lin-low), or by using fluorescent vital dyes such as Hoescht 33342. Human hematopoietic stem cells have been isolated primarily through their expression of the marker CD 34, lack of lineage markers, and low expression of Thy1.

b. Endothelial Progenitor Cells (EPC)

Postnatal bone marrow contains a subtype of cells called endothelial progenitor cells, which have the capacity to migrate to the peripheral circulation and to differentiate into mature endothelial cells. There is increasing evidence that endothelial progenitors primarily arise from hematopoietic stem cells. The exact characterization of endothelial progenitor cells remains to be defined. Both hematopoietic and endothelial precursors express common epitopes that include Flk-1, Tie-2, CD34, Sca-1, c-Kit, thrombomodulin, GATA-4, GATA-6, and others (Hatzopoulous et al. Development-Supplement 125(8): 1457-1468 (1998); Harraz et al. Stem Cells 19(4): 304-312 (2001)). The expression of VEGF-2R or KDR receptor defines a subset of CD34/CD38 positive cells, some of which have the ability to differentiate along an endothelial lineage. AC133 (CD133) is a more primitive hematopoietic stem cell marker that is expressed on the majority of CD34+ cells, but unlike CD34, its expression is lost during maturation of EPCs, thus allowing an earlier and perhaps more precise identification of EPCs. However, AC133-negative cells and CD34-negative cells selected from peripheral blood, as well as the non-hematopoietic multipotent stem cell, will also form endothelial-like colonies and differentiate to produce cells expressing mature endothelial cell markers (Raffi et al. Seminars in Cell & Developmental Biology 13(1): 61-67 (2002); Bussolino et al. Nature 337: 471-473 (1989)). The cells may be isolated by cell sorting using flow cytometry after labeling peripheral blood samples for CD34, AC133, and KDR epitopes. Because of the limitations of cell sorting technologies in identifying EPCs within the mononuclear cell population, and the relative paucity of these cells (0.01%) in peripheral blood, functional assays have been developed so that EPCs can now be isolated and reproducibly grown from peripheral blood samples or from buffy coats. In addition to cell sorting techniques, peripheral blood EPC count may be measured by cell culture.

IV. Applications

The principle application of the combi-chips is to rapidly screen for the conditions most useful in attachment, growth, propagation and differentiation of one or more cell types, especially stem cells and progenitor cells. The selection of such conditions is highly cell specific and difficult to predict. Application of the cells to a single substrate having many micro-domains provides a means for rapidly determining which conditions and materials are most conducive to a desired outcome. Substates have regions varying in composition, method of formation (and therefore structural features), and composition and concentration of bioactive molecules adhered thereto.

Once the preferred conditions have been selected, substrates can be prepared on a larger scale and used to direct differentiation of stem cells adhering to engineered substrates to lineage-specific progeny.

The substrates can also be used to test the effect of molecules on the cells under the different conditions within the separate microdomains and to test their use for the treatment or replacement of tissues and organs damaged by disease or chemotherapy.

It is likely that substrates that induce growth signals in stem cells are more compatible with stem cell differentiation, and that lineage commitment depends upon the presence of specific growth factors in the micro-environment. In a preferred embodiment, the stem cells adhering to the engineered substrates are directed to differentiate into endothelial progeny due to their potential to promote the formation of vascular structures that could rescue and render functional any ischemic organ, graft, or tissue engineered construct. For example, stem cells collected from animals (e.g., genetically engineered mice, pigs), or from human bone marrow donors may be differentiated into EPC and used in re-populating appropriate tissue compartments. The ex vivo expanded cells may be re-injected in patients to improve oxygenation in tissue rendered ischemic due to arterial obstruction (e.g., myocardial infarction, stroke) through enhanced formation of collaterals. Also, EPC may be used to promote regeneration of the endothelial lining of diseased arteries damaged by stroke-induced ischemia.

The specific information derived from the combinatorial libraries permits the design of two-dimensional and/or three-dimensional scaffolds for in vivo application. For example, once an optimal surface “formulation” is discovered combinatorially, the formulation can be used to fabricate laboratory- or clinical-scale two-dimensional surfaces for the in vitro expansion and differentiation of stem cells in therapeutic quantities. In this context, a surface formulation would consist of the composition of components (polymers, proteins, polypeptides, minerals, and other nutrients) and the mixing and annealing (thermal treatment) conditions. Likewise, three-dimensional porous scaffolds can also be fabricated for tissue engineering applications by integrating the surface formulation, discovered combinatorially, into the existing procedure for creating porosity. For example, the porosity may be introduced via the common method of salt leaching, in which salt particles are dispersed into a 3D polymer sample prepared at the optimal formulation composition. Prior to leaching the salt to create pores, the sample would be thermally annealed to create the desired surface microstructure at the interface between the polymer and salt. When the salt is leached, the scaffold contains pores of a desired size in which the surface of the pores expresses the optimal surface physical and chemical features. Such scaffolds may be useful, for example, in tissue engineering or bone regeneration.

The present invention will be further understood by reference to the following non-limiting examples.

### EXAMPLE 1

### Preparation of Composition Gradient Libraries

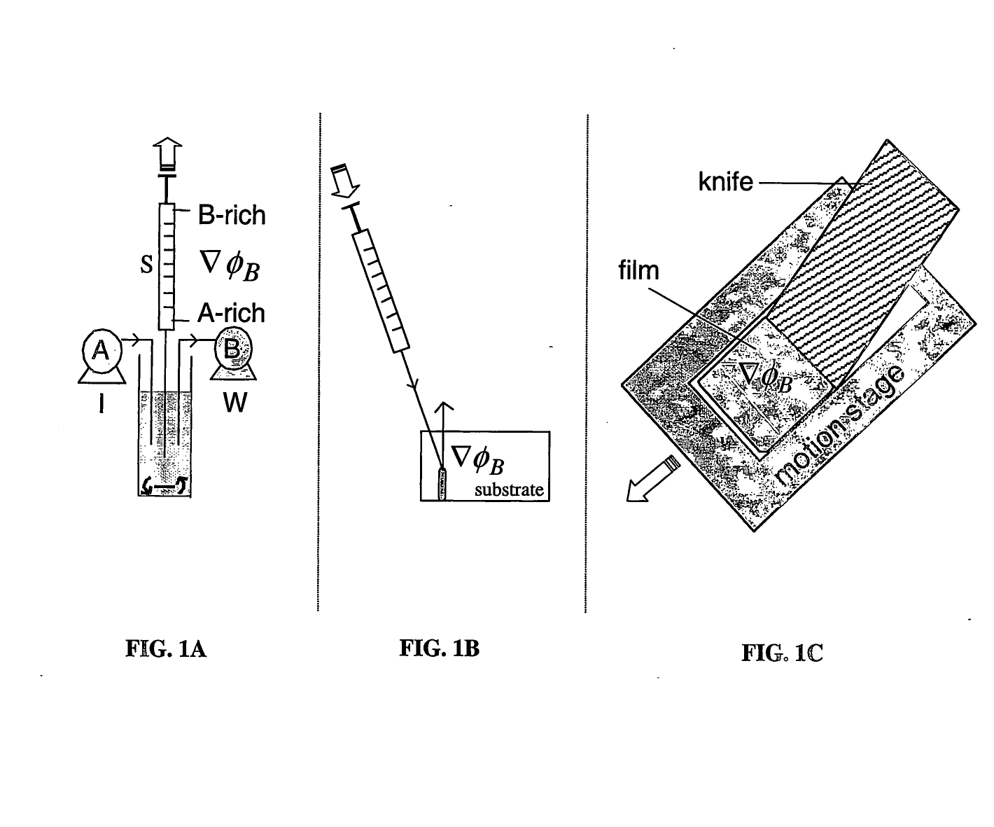

Poly(D,L-lactide) PDLA (Alkermes, Medisorb 100DL, M_{w}=127000, M_{w}/Mₙ=1.56) and poly(ε-caprolactone) (PCL) (Aldrich, M_{w}=114,000, M_{w}/Mₙ=1.43) were obtained from commercial suppliers. Each T-φ combinatorial library consists of a polymer film on a clean silicon (Si—H/Si) wafer, with dimensions of 25×30 mm along orthogonal gradients in φ_{PCL} and annealing T. The composition gradient deposition process, shown in FIG. 1, is applicable to a wide range of polymer blends. A PCL solution in CHCl₃ is pumped into a mixing vial initially containing a PDLA solution while the mixture is withdrawn, resulting in a linear increase in PCL composition in the vial. A third automated syringe extracts the φ-gradient solution from the vial and deposits it as a thin stripe on the substrate. A knife-edge coater spreads the liquid as a film orthogonal to the composition gradient. FTIR spectroscopy was used to measure compositions to within mass fraction 4% on libraries by obtaining spectra in the C—H stretch regime (2700 to 3100) cm⁻¹. The library deposition technique has been well characterized in Meredith, J. C. et al. J. Biomed. Mater. Res. 66: 483-490 (2003) and Meredith, J. C. and Amis, E. J. Macromol. Chem. Phys. 201: 733-739 (2000).

PDLA/PCL blends exhibit lower critical solution temperature (LCST) phase behavior, where PDLA and PCL separate at T>86° C. LCST phase transition allows for the adjustment of microstructure and roughness via composition (φ), processing T, and processing time. When the blend is quenched back to room temperature, the two-phase structure is preserved due to the glass transition of PDLA (55° C.) and crystallization of PCL (T<60° C.).

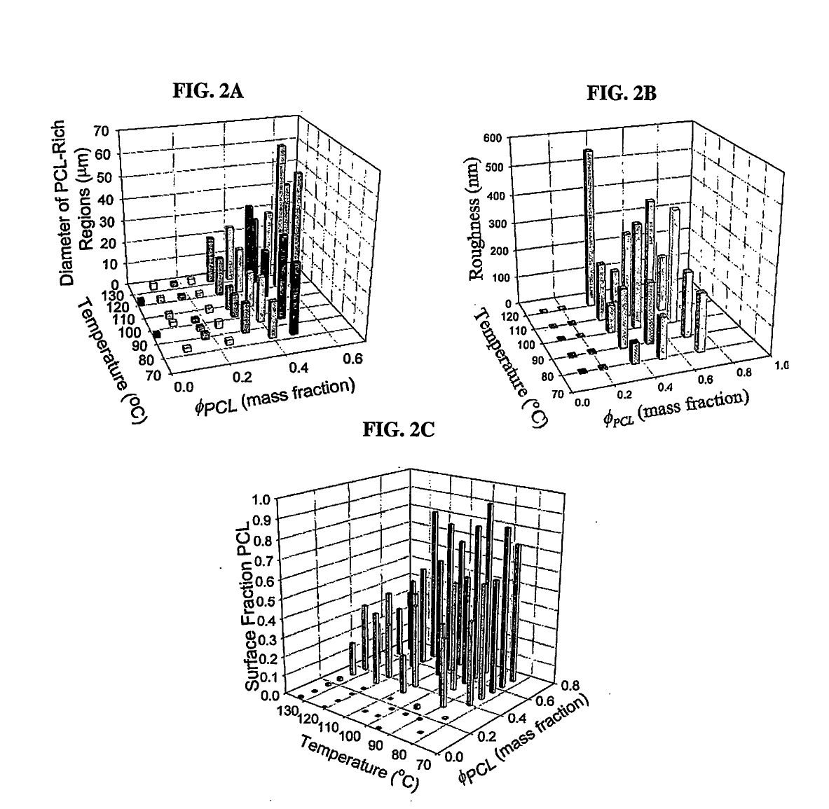

The primary surface differences between regions within and outside the LCST are the microstructure (lateral distribution of chemically distinct domains) and surface roughness. Atomic force micrographs (AFM) and optical images from libraries were used to quantify the surface roughness and microstructure sizes. The diameter of PCL-rich regimes, d_{PCL}, increases with both φ_{PCL} and T (FIG. 2A), and covers a range of (0.2<d_{PCL}<60)μm. Beyond d_{PCL}=60 μm the PCL phase becomes continuous with dispersed PDLA droplets. Phase separation also induces changes in the roughness of the surface, which are attributed to surface tension differences between chemically distinct domains and between crystalline and amorphous PCL. With a few exceptions root-mean-square (rms) surface roughness, R, increases with both φ_{PCL} and T over a range of (2<R<500) nm (FIG. 2B). FIG. 2C shows surface fraction of PCL-rich phase, as measured by polarized optical microscopy, as a function of initial composition of PCL in the blend and annealing temperature. It was assumed that the PCL-rich phase was the only phase that exhibited birefringence under polarized light.

### EXAMPLE 2

### Isolation, Fractionation, and Preparation of Murine and Human Adult Hematopoietic Stem Cells

a. Isolation and Fractionation of Murine Adult Hematopoietic Stem Cells

Bone marrow was harvested by flushing isolated femurs and tibias of donor mice with RPMI 1640 supplemented with 1% FBS, 1%-l-glutamine, and penicillin/streptomycin (“transplant medium”). Bone marrow mononuclear cells (MNC) were then isolated by centrifugation in Ficoll-Hypaque density separation medium (ρ=1.077 g/ml). MNC were incubated with a panel of lineage specific biotinylated monoclonal antibodies followed by secondary antibodies bound to paramagnetic beads, and fractionated on a MidiMACS column to obtain a HSC-enriched population by negative selection. The resulting fraction was further purified by cell sorting via a FACSVantage flow cytometer to obtain specific subpopulations of HSC (e.g., c-Kit⁺Sca-1⁺1lin⁻).

b. Preparation of Adult Human Hematopoietic Stem Cells

Blood or bone marrow samples were obtained after informed consent from CSF-mobilized patients. Following centrifugation of WBC suspensions on Ficoll-Hypaque to remove dead cells, CD 34⁺ were isolated by positive selection using immunomagnetic bead fractionation on a MiniMACS magnet system (Miltenyi Biotec). The CD 34⁺ enriched fraction was then further purified by high-speed FACS sorting using a FACSVantage cell sorter.

### EXAMPLE 3

### Determination of the Biological Effects of Stem Cell Adhesion to Combinatorial Surfaces

Mouse and human stem cells were used to determine the differential adhesive properties, of combinatorial surfaces, for stem cells and whether specific regions of combinatorial surfaces enhance viability, proliferation and differentiation of these cells.

a. Cell Culture

The pre-annealed T, φ libraries were prepared and quenched to room temperature and sterilized in 70% ethanol in a laminar flow hood. Murine whole bone marrow or purified bone marrow MNC or human CD 34⁺ stem cells were cultured for 4-7 days directly on the combinatorial surfaces in minimal essential medium (MEM) supplemented with 10% fetal bovine serum (FBS) and 50 ng/ml vascular endothelial growth factor (VEGF) to promote survival and differentiation of endothelial progenitors. In some experiments, bone marrow cells were cultured with one or more additional cytokines (e.g. SCF, IL-3) to determine whether there was a synergistic effect between cytokine stimulation and polymer surface properties. Cells were cultured in parallel on glass coverslips and on tissue culture polystyrene as uniform surface controls for comparison to two-dimensional libraries.

b. Pre-Coating the Combinatorial Libraries with Extracellular Matrix (ECM) Proteins Including Fibronectin, Collagen IV, and Laminin.

Combinatorial polymer libraries were incubated with 5 μg/cm² of ECM proteins for 1 hr at 37° C., then blocked with 1% BSA for 1 hr at 37° C. Comparison of pre-coated, and uncoated libraries provides an indication of whether microstructure affects cell function directly, or only secondarily through its influence on ECM protein conformation.

c. Cell Staining and Characterization

Total cell adhesion and viability were assessed by staining cultures with calcein AM and ethidium bromide for visualization by fluorescence microscopy (Live-Dead assay, Molecular Probes). For confirmation of endothelial cell (EC) identity and quantification of differential adhesion of ECs/EPCs and white blood cells (WBCs), double labeling immunocytochemistry for CD31 (EC marker) and CD 45(WBC-specific marker) was performed using secondary antibodies labeled with Rhodamine Red-X and Alexa Fluor 466, respectively.

d. Analysis of Combinatorial Polymer Libraries

Fluorescence images were acquired on a 3×3 mm grid to cover the entire parameter space of the combinatorial library, so that cell density and viability could be correlated to polymer composition and material properties. Matlab software was used to generate a density contour map for the entire surface from this data. For initial rapid screening of polymer libraries, the adherent cells were stained with Cy5-labeled secondary antibodies and counterstained with SYBR Green I, then scanned with the 473 nm and 633 nm excitation wavelengths on a Fuji FLA3000 phosphoimager. This approach permits rapid visualization of the entire surface area.

e. Investigation of Stem Cell Differentiation

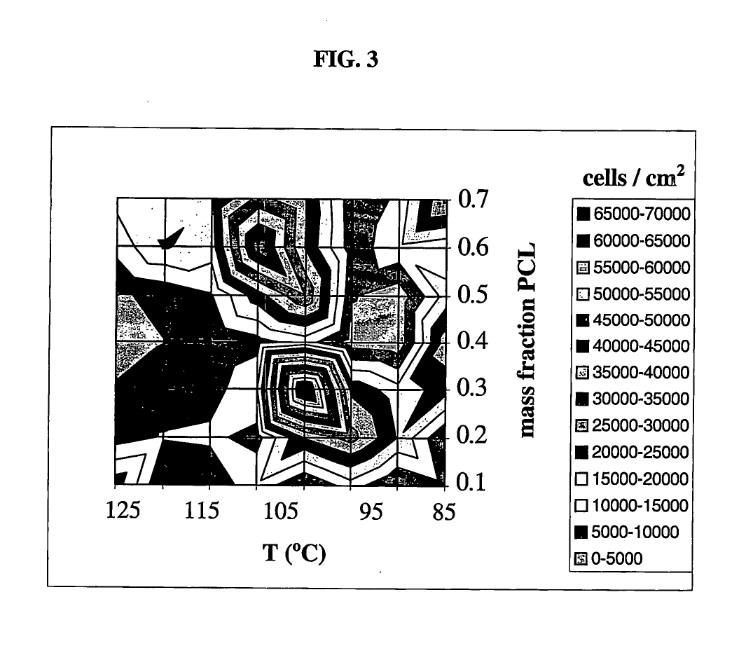

Mouse bone marrow (BM) cells cultured on combinatorial chips for up to four days were stained using immunocytochemistry for CD 31 (endothelial cell-specific marker). Fluorescence images were acquired on a grid that covers the entire T, φ space of the combinatorial library, so that cell density and viability could be correlated to polymer composition and material properties. As shown in FIG. 3, these tests demonstrate differential adhesion and viability of mouse BM cells cultured on polymer libraries. The differences in adherent cell morphology as a function of position suggest that BM cells belonging to different lineages adhere preferentially to areas having specific surface properties. These results illustrate the feasibility of the high-throughput method for culturing cells directly on combinatorial libraries. The effect of coating chips with natural extracellular substrates was also examined. For example, stem cells demonstrate enhanced differentiation towards the endothelial cell lineage of cells when cultured for 4 days on a collagen IV-coated combinatorial polymer film.

The effects upon differentiation towards the EC lineage by using bone marrow cells isolated from mice genetically engineered to express green fluorescent protein under the control of the Tie-2 endothelial-specific promoter were tested. An advantage to this approach is that one can monitor the differentiation of live cells (no additional processing that harms the cell is required), and track them in vivo (for example, to explore homing and integration of stem cells injected after controlled manipulation in vitro).

We claim:

1. A method of inducing stem cell adhesion, survival, proliferation, or differentiation comprising contacting stem or undifferentiated cells with a combinatorial substrate library comprising discrete regions varying in surface composition, microstructure, and molecules bound thereto.

2. The method of claim 1 wherein discrete regions are coated with one or more bioactive molecules.

3. The method of claim 2 wherein the bioactive molecules are selected from the group consisting of growth factors, extracellular matrix molecules, and cytokines.

4. The method of claim 1 wherein the substrate is a polymer or polymer blend.

5. The method of claim 4 wherein the polymer or polymer blend is biodegradable.

6. The method of claim 1 further comprising identifying discrete regions inducing preferred properties in the cells adhered thereto.

7. The method of claim 1 further comprising differentiating the stem cells into specific lineages following contact with the substrate.

8. The method of claim 7 wherein a specific lineage is an endothelial lineage.

9. A combinatorial substrate library comprising discrete regions varying in surface composition, microstructure, and molecules bound thereto having stem cells or undifferentiated cells adhered thereto.

10. The combinatorial substrate library of claim 9, wherein one or more discrete regions are coated with one or more bioactive molecules.

11. The combinatorial substrate library of claim 10 wherein the bioactive molecules are selected from the group consisting of growth factors, extracellular matrix molecules, and cytokines.

12. The combinatorial substrate library of claim 9, wherein the substrate is a polymer or polymer blend.

13. The combinatorial substrate library of claim 12, wherein the polymer or polymer blend is biodegradable.
